(19) United States
(12) Patent Application Publication
(10) Pub. No.: US 2022/0114674 A1
(43) Pub. Date: Apr. 14, 2022

(54) HEALTH LAB DATA MODEL FOR RISK ASSESSMENT

(71) Applicant: Hi.Q, Inc., Mountain View, CA (US)

(72) Inventors: Ryan Hinchey, Mountain View, CA (US); Munjal Shah, Mountain View, CA (US); Gaurav Suri, Mountain View, CA (US); Kun Huang, Mountain View, CA (US); Kurt Roots, Mountain View, CA (US); Bill Moore, Mountain View, CA (US); Janel Obenchain, Mountain View, CA (US)

(21) Appl. No.: 17/496,478

(22) Filed: Oct. 7, 2021

Related U.S. Application Data
(60) Provisional application No. 63/089,697, filed on Oct. 9, 2020.

(51) Int. Cl. G06Q 40/08; G16H 50/30; G16H 10/40; G06F 16/2458

(52) U.S. Cl. CPC G06Q 40/08; G06F 16/2468; G16H 10/40; G16H 50/30

(57) ABSTRACT

A computing system can implement a risk-assessment service through execution of a health lab data classifier model that classifies a prospective client's clinical health lab data, comprising a set of health aspect levels, within a set of health aspect ranges. When the set of health aspect levels of the prospective client are within a set of health aspect ranges, the computing system can generate one or more risk-mitigation packages at a particular tier of the risk-assessment service.

### CROSS REFERENCE TO RELATED APPLICATION

This application claims the benefit of priority to U.S. Provisional Application No. 63/089,697, filed on Oct. 9, 2020, which is hereby incorporated by reference in its entirety.

### BACKGROUND

Risk assessment techniques have typically involved generalizations with respect to factors such as health, age, income, assets, liabilities, etc. With the advent of big data and computer modeling techniques, risk assessment has become more granular, with improvements in such techniques providing more precision and efficiency for consumers of risk mitigation products.

### BRIEF DESCRIPTION OF THE DRAWINGS

The disclosure herein is illustrated by way of example, and not by way of limitation, in the figures of the accompanying drawings in which like reference numerals refer to similar elements, and in which:

FIG. 1 illustrates a network-based computing system executing filtering and verification techniques for determining individualized health or mortality risks for prospective clients, according to examples described herein;

FIG. 2 illustrates client computing device for engaging with a risk-assessment service provided by the network-based computing system, according to examples described herein;

FIG. 3 is a flow chart describing an example method of verifying self-assessment responses from users, according to example described herein;

FIG. 4 is a flow chart describing an example method of executing a health data model in response to a client filtering process, according to various examples; and

FIG. 5 is a block diagram that illustrates a computer system upon which examples described herein may be implemented.

### DETAILED DESCRIPTION

A network-based computing system can implement a health risk assessment service that executes a data classifier model using health lab information of potential clients to provide more granular risk segmentation for risk-mitigation products (e.g., provide one or more additional tiers) that can result in more efficient and higher quality health care for qualifiers of these segmented tiers. As senior citizens get older, health care becomes more and more expensive, which can result in higher health care premiums and/or deductibles that need to be paid in order to for these seniors to receive health care. Through advances in machine learning and model training technology, a computing system can provide increasingly greater accuracy in predicting health risks and/or mortality risks on the individual level. As computer technology advances, these computational models can more granularly determine health risks and life expectancy, and in turn, provide additional underwriting classes or individually tailor risk-mitigation packages to potential clients.

Past methods of classifying individuals into underwriting classes have involved the use of such factors as age, height, weight, and gender to make a loose prediction of risk. In some aspects, the individual provides self-reporting information (e.g., via a questionnaire) to enable a health service provider to determine family health history (e.g., cancer risk, heart disease risk, etc.). However, without detailed parsing of historical records of the potential client, risk-mitigation providers are left unable to determine whether the potential client is being untruthful. Furthermore, typically, senior patients are routinely provided with clinical lab tests involving blood samples, urine samples, other bodily fluid samples, and or body tissue samples to determine any current health risks or abnormalities. These clinical tests can provide detailed information concerning health risks such as diabetes, kidney disease, cardiovascular disease, liver function/disease, frailty risk, and the like.

According to examples described herein, a computing system can leverage the use of a trained classifier model to accurately determine current health and/or mortality risks and classify individuals into underwriting classes accordingly. In certain examples, the ability to run clinical lab data through such a computational model can enable more accurately priced risk-mitigation products that have the effect of driving down costs for healthy seniors. In various examples, the computing system or a human operator associated with the health risk assessment service can receive an inquiry from a prospective client to determine a qualification for a particular risk-mitigation product (e.g., an insurance product), package, or underwriting class. In response, the computing system or human operator can provide a set of self-reporting queries to allow the prospective client to provide a set of corresponding self-assessment responses. Such responses can include basic information such as the prospective client's age, height, weight, gender, home address, whether the prospective client is a smoker, etc.

In certain examples, the responses can be received by the computing system or manually entered by the human operator. In either case, the computing system can execute verification logic to determine the truthfulness of the prospective client's self-assessment responses, and whether the prospective client satisfies a set of initial health risk criteria. For example, in some aspects, the computing system can access public profile data of the prospective client to determine truthfulness concerning the prospective client's age or height. In further examples, the computing system can receive location data from a positioning system of the client's computing device. The computing system can store map data indicating the locations of various health care providers, such as clinics, hospitals, nursing homes, hospice care facilities, and the like. When the prospective client provides self-reporting of a current address, the computing system can verify a truthfulness of the current address by correlating the client's response with the received location data.

In various implementations, the computing system or human operator can query the prospective client of whether the client currently resides in a nursing home, assisted living facility, hospice care facility or other professional care facility.

Upon receiving a response, the computing system can verify the prospective client's response using the location data that the client had self-reported or from the client's computing device and the map database of health care providers. If the location data indicates that the prospective client is not truthful, then the computing system or human operator can decline the prospective client from further engagement (e.g., declining the prospective client from qualifying for a specific Medicare supplement policy). However, when truthfulness is verified by the verification logic, and the criteria are met, the computing system can qualify the prospective client for an initial class of risk mitigation products or packages, and/or can advance to a next step of the filtering process.

As such, execution of the verification logic can enable the computing system to filter the prospective clients for further engagement. In various examples, the filtering process may proceed to an access request of the prospective client's historical prescription data and/or medical claims data. In certain examples, the prospective client may interact with the computing system via a website or interactive user interface of a health service application executing on the prospective client's computing device. In one example, the website or interactive user interface can provide an icon or graphical button that, when selected, enables the computing system to synchronize with or access one or more health accounts of the prospective client through a single application programming interface (“API”). Once synchronized or access-permitted, the computing system can obtain the prospective client's historical prescription information and medical claims history to determine whether the prospective client satisfies a set of historical health criteria. If so, the computing system can qualify the prospective client for a specified tier of risk-mitigation products or packages, or advance to a next step of the filtering process.

According to various examples, the computing system can transmit an access request for the prospective client's clinical lab data. In variations, the prospective client's clinical lab data can be received as a screenshot of a client's medical records or electronic health records, and automatically parsed and inputted by the computing system (e.g., via optical character recognition techniques) or manually inputted by a human operator. As described herein, these data sets can provide the computing system with blood work and or bodily fluid sample information (collectively “health aspect levels”) such as total cholesterol level, high-density lipoprotein (HDL) level, HDL ratio, triglycerides level, creatinine level, albumin level, alkaline phosphatase level, glucose level, hemoglobin level, hematocrit level, A1C level, liver function levels (e.g., ALT and AST), total bilirubin levels, BUN/creatinine ratio, urea nitrogen (BUN) level, albumin/globulin ratio, eGFR (estimated glomerular filtration rate), bilirubin level, vitamin D levels, C-reactive protein level, troponin I level, potassium level, hepatitis C condition, factors arising from a urine protein test, cystatin C level, etc. In certain examples, the computing system can access multiple blood test results of the prospective client to determine the client's sample information over time and any improvements or detriments.

Using historical sample data from a control group of past patients, and correlating those data to the historical medical claims and prescription information of the past patients, the computing system can execute a health lab data model to generate an ideal range or set of ranges for each clinical lab data set (e.g., a healthy cholesterol range, HDL ratio, creatinine level, etc.). In further examples, the computing system can further use additional historical data, such as historical incurred claims information by patients, earned premiums on Medicare supplements or other risk-mitigation products, and the like to determine loss ratios for training the health lab data model. Based on the historical data, these ideal ranges correlate to prospective clients that will qualify for a top tier or underwriting class that can, for example, reward those prospective clients for living healthy and active lifestyles with higher quality and/or lower cost risk-mitigation packages. Conversely, prospective clients having health aspect levels that fall outside these ideal ranges (e.g., those who are found to be diabetic, have unhealthy cholesterol levels, overweight, smokers, etc.) may not qualify for a risk-mitigation tier, or my result in a tier that involves higher costs. As provided herein, the health lab data model can be trained to determine the ideal ranges for each clinical lab data set as a proprietary and customized set of health data ranges (e.g., and/or to minimize loss ratio(s)). In doing so, the health lab data model can further correlate current medical claim data and prescription information of a current set of health service clients—using their clinical lab data that are within the set of ideal ranges—with the current medical claim data and prescription information of patients in general to compare loss ratios between the two groups.

In further examples, the computing system can receive activity data from an activity monitoring device (e.g., a wearable computing device, such as a wrist-worn device or a chest-worn device, the client's mobile computing device, or an embedded exercise monitoring device on exercise equipment, like an exercise bicycle) that can indicate information such as the prospective client's heart rate, the number of steps taken throughout the day, calories burned, sleep scores, an average and/or peak walking or running pace of the prospective client, an average and/or peak swimming pace, an exercise distance, etc. The health lab data model can further process the activity data of the prospective client (e.g., for a single activity, or for a previous month or multiple previous months) to determine an activity level and/or sleep level of the prospective client as compared to an activity range for humans in general, or humans of similar age and gender. The computing system can classify the prospective client in each aspect of the activity data and/or sleep score data (e.g., 85th percentile in steps taken per day) and incorporate the prospective client's activity and/or sleep levels with the prospective client's clinical lab data set information in determining a mortality or morbidity score for the prospective client.

In still further examples, the computing system can further provide the prospective client with access to a cognitive assessment test (“CAT”). The CAT may be taken remotely and electronically through a website or via a user interface of the health risk assessment application. In variations, the CAT may be communicated to the prospective client verbally (e.g., over a phone) while the prospective client makes verbal responses. The CAT can be highly correlative of cognitive risk assessment through tuning of question by a control base of individuals of which cognitive health outcomes are known (e.g., those that have since developed dementia). CAT responses can comprise each control base individual's response to each CAT question or assertion. The CAT can comprise filtered questions and assertions (e.g., a multiple-choice quiz) that test various cognitive health metrics of the users, such as spatial reasoning, motor function, word-finding and language processing ability, and time to completion for each question/assertion as well as the overcall CAT. As such, CAT performance of the individuals in the control base can enable the computing system to generally measure the cognitive well-being of the control base individuals (e.g., comprised of senior citizens) by digitally assessing memory and recall, word finding abilities, reaction time and motor function, and the like.

The prospective client may take the CAT and provide responses, which can be analyzed through execution of the accurate cognitive correlation model to generate a set of cognitive risk scores for the prospective client. As provided herein, this set of cognitive risk scores can correlate directly with vehicular accident risk morbidity risk and/or mortality risk, and may be used by the computing system to generate a risk mitigation policy or underwriting class for the user. In certain examples, the risk mitigation policy can be individualized for the user or can be a tiered class of risk mitigation policies for a like cluster of users (e.g., an underwriting class) having similar cognitive risk scores.

Examples described herein achieve a technical effect of utilizing big data and machine learning techniques to more accurately provide individualized health risk assessments for prospective clients, particularly for elderly individuals, and determining mortality risk on a case by case basis. On a practical level, elderly individuals must typically acquire risk mitigation products at significantly higher rates that steadily increase over time due to an assumption that mortality, morbidity and/or driving accident risk generally increases similarly for everybody as elderly people grow older. However, for certain healthy elderly individuals, mortality, morbidity and/or driving accident risk progresses far more slowly than others, yet they must still attain risk mitigation products (e.g., life insurance, auto insurance, Medicare Supplement, etc.) at higher rates due to such industry generalizations. The techniques described herein can identify these low risk, elderly individuals with low mortality, morbidity and/or driving accident risk through computational analysis of the individual's truthfulness, health lab and/or activity data, and/or cognitive performance to provide better coverage and/or lower rates for risk mitigation products.

As used herein, a computing device refers to devices corresponding to desktop computers, cellular devices or smartphones, personal digital assistants (PDAs), laptop computers, virtual reality (VR) or augmented reality (AR) headsets, tablet devices, television (IP Television), etc., that can provide network connectivity and processing resources for communicating with the system over a network. A computing device can also correspond to custom hardware, in-vehicle devices, or on-board computers, etc. The computing device can also operate a designated application configured to communicate with the network service.

One or more examples described herein provide that methods, techniques, and actions performed by a computing device are performed programmatically, or as a computer-implemented method. Programmatically, as used herein, means through the use of code or computer-executable instructions. These instructions can be stored in one or more memory resources of the computing device. A programmatically performed step may or may not be automatic.

One or more examples described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines.

Some examples described herein can generally require the use of computing devices, including processing and memory resources. For example, one or more examples described herein may be implemented, in whole or in part, on computing devices such as servers, desktop computers, cellular or smartphones, personal digital assistants (e.g., PDAs), laptop computers, VR or AR devices, printers, digital picture frames, network equipment (e.g., routers) and tablet devices. Memory, processing, and network resources may all be used in connection with the establishment, use, or performance of any example described herein (including with the performance of any method or with the implementation of any system).

Furthermore, one or more examples described herein may be implemented through the use of instructions that are executable by one or more processors. These instructions may be carried on a computer-readable medium. Machines shown or described with figures below provide examples of processing resources and computer-readable mediums on which instructions for implementing examples disclosed herein can be carried and/or executed. In particular, the numerous machines shown with examples of the invention include processors and various forms of memory for holding data and instructions. Examples of computer-readable mediums include permanent memory storage devices, such as hard drives on personal computers or servers. Other examples of computer storage mediums include portable storage units, such as CD or DVD units, flash memory (such as carried on smartphones, multifunctional devices or tablets), and magnetic memory. Computers, terminals, network enabled devices (e.g., mobile devices, such as cell phones) are all examples of machines and devices that utilize processors, memory, and instructions stored on computer-readable mediums. Additionally, examples may be implemented in the form of computer-programs, or a computer usable carrier medium capable of carrying such a program.

System Description

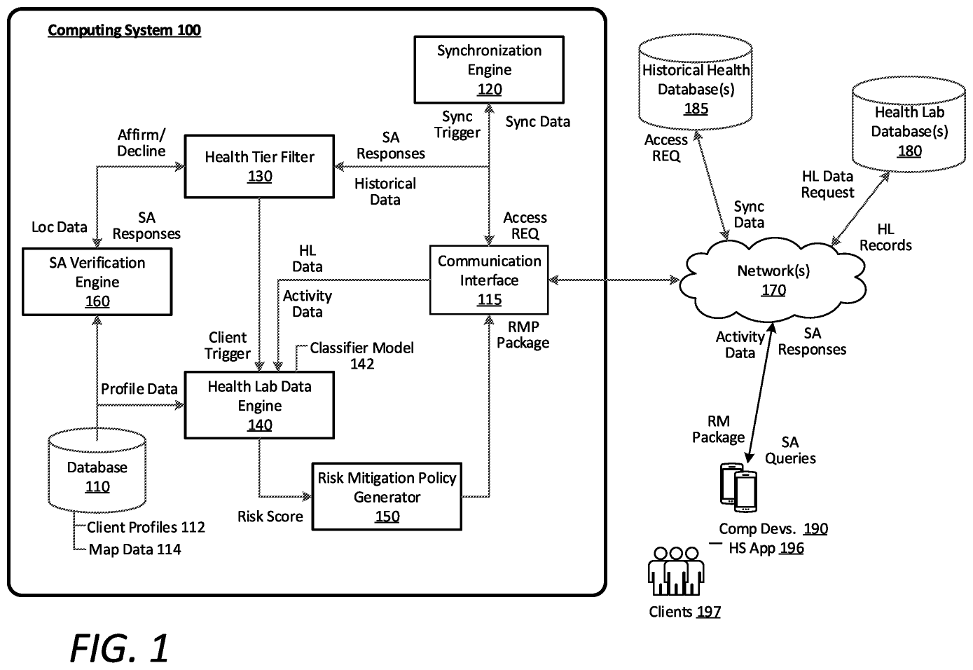

FIG. 1 illustrates a network-based computing system 100 executing filtering and verification techniques for determining individualized health risks for prospective clients 197, according to examples described herein. In various examples, the computing system 100 includes a communication interface 115 to communicate over one or more networks 170 with computing devices 190 of clients 197 of a health risk-assessment service provided via the computing system 100. The computing system 100 can include a health tier filter 130 that can receive an initial health service inquiry from the computing device 190 of a particular client 197. In certain examples, the computing device 190 can comprise a personal computer in which the client 197 accesses the computing system 100 via a dedicated health service website. Additionally or alternatively, the client 197 can communicate with the computing system 100 via an interactive user interface through an executing health service application 196 on the computing device 190.

Based on the initial inquiry, the health tier filter 130 can provide the client 197 with a set of self-assessment queries to determine the client's 197 age, weight, height, demographic information, rural versus urban home information, a home address, gender or sex, smoker status, whether the prospective client 197 has a spouse or roommate, and any current or historical health risks or problems. In various examples, the client 197 can provide self-assessment responses to the health tier filter 130 indicating such information. In certain implementations, the computing system 100 can include a self-assessment verification engine 160 that correlates the client's 197 self-assessment responses with known information pertaining to the client 197. In one example, this known information can comprise location data from the client's 197 computing device 190 that indicates a current location of the client 197. Thus, upon receiving a self-assessment response indicating that the client 197 lives at a current address, the self-assessment verification engine 160 can compare the current location of the client 197 with map data 114 (e.g., from a local database 100 of known health care locations, such as nursing homes and hospice care facilities) to determine whether the client 197 is being truthful.

In variations, the verification engine 160 can receive input indicating the prospective client's 197 address (e.g., via a questionnaire or vocalized). Based on the input, the verification engine 160 can utilize a directory of known addresses and/or locations of nursing homes, assisted care facilities, etc., in the database 110 to determine whether a match exists. For certain locations (e.g., rural areas), the verification engine 160 can infer whether a match exists based on a threshold proximity of the inputted address with the known addresses and/or locations of such facilities. For example, the verification engine 160 can execute fuzzy matching logic that can incorporate truth values or ranges may result in a match even if the inputted address and the known locations do not match exactly.

Based on the client's 197 location, the self-assessment verification engine 160 can provide either an affirmation that the client 197 is being truthful or decline the client's 197 truthfulness if the inputted location by the client 197 does not align with the client's 197 current location or inputted location. For example, the self-assessment queries provided to the client 197 can ask whether the client 197 currently resides in a nursing home. Based on the client's 197 answer and the location data from the client's 197 computing device 190, the self-assessment verification engine 160 can perform a lookup in the map data 114 of the locations of nursing homes within a proximity of the client's 197 current location. If a nursing home location aligns with the current location of the client 197, the self-assessment verification engine 160 can decline the client 197 from further advancement in the filtering process.

The self-assessment verification engine 160 can further determine truthfulness using profile data of the client 197—either from a local database 110 of client profiles 112 or via access to one or more public records of the client 197—to verify self-assessment responses from the client 197. For example, the self-assessment verification engine 160 can determine a truthfulness of the client 197 with respect to a statement of age, weight, and/or current health conditions using publicly available information of the client 197. For affirmed clients 197, the health tier filter 130 can then provide a synchronization or access request icon or graphical button on the display screen of the website or health service application 196 (e.g., displayed in conjunction with consent or waiver information that the prospective client 197 is requested to read or listen to and provide their consent). The health tier filter 130 may further provide a request for the client 197 to select the icon or graphical button, which can cause a synchronization trigger to enable a synchronization engine 120 of the computing system 100 to synchronize with other applications on the computing device 190 of the client 197, or provide access to a health-related history of the client 197.

Additionally or alternatively, a human operator can engage the prospective client 197 via a telephone discussion or other voice interaction, and can be provided with a verbal consent inquiry (e.g., a HIPAA waiver of authorization). he prospective client 197 can provide consent accordingly. Upon receiving consent, the synchronization engine 120 can access historical data of the prospective client 197 from one or more third-party systems or databases (e.g., lab data, prescription data, and/or medical claims data). Using this historical data, the health tier filter 130 can translate or process the data to determine a risk score for the prospective client that corresponds to a particular underwriting class or a rejection of coverage.

In one approach, the prospective client 197 is prompted to log into a health-related account (e.g., the client's 197 myMedicare.gov account) and provide authorization or consent to enable the health tier filter 130 to access data associated with the account. Upon receiving the trigger, the health-tier filter 130 can obtain prescription data and fill information, medical claims information (e.g., including ICD, CPT and HCPCS codes along with claim amounts, dates of service, and an identifier of the doctor performing the service). In further examples, this authorization can also enable the computing system 100 to access the health lab data, as described below.

In certain aspects, upon receiving the synchronization trigger that provides the necessary permissions to access the client's 197 historical information, the synchronization engine 120 can access the client's accounts (e.g., username and password sensitive accounts) with one or more health service provider(s) to receive sync data comprising the client's 197 historical prescription data and medical claims and diagnosis data. These data sets can indicate how many and which types of prescription medications the client 197 currently and historically uses along with the client's 197 adherence to the prescriptions as well as the client's 197 history of medical insurance claims, medical diagnosis (e.g. ICD-10 code C80.1 for Malignant (primary) neoplasm), and/or hospital visits (e.g., emergency visits, scheduled checkups, and the like). The synchronization engine 120 can parse the client's 197 historical information to provide the health tier filter 130 with the relevant prescription, medical claims and/or medical diagnosis information, which the health tier filter 130 can classify in terms of health risk (e.g., mortality risk, health risk, disease risk, etc.).

Based on the client's historical health and prescription data, the health tier filter 130 can determine whether the client 197 qualifies for a certain tier or underwriting class of the risk-mitigation service (e.g., providing access to lower cost risk-mitigation packages). If so, the health tier filter 130 can provide a client trigger to a health lab data engine 140 of the computing system 100 to determine whether the client 197 qualifies for a top tier of the risk-mitigation service (e.g., lowest cost and/or highest quality risk mitigation packages). In various implementations, once prospective clients 197 are filtered through the preceding processes, the health lab engine 140 can be initiated to access clinical health lab data of each client 197 from one or more health lab databases 180. In certain examples, the health lab data can be accessed via a graphical icon trigger as described above, which can provide the necessary permissions from the client 197. The health lab data engine 140 can execute a classifier model 142 (e.g., trained computer model, that comprises customize ranges for each health lab data set that identify which clients 197 qualify for this highest tier.

As described herein, the data sets from the health lab database(s) 180 can provide the computing system with blood work and or bodily fluid sample information such as total cholesterol level, high-density lipoprotein (HDL) level, HDL ratio, triglycerides level, creatinine level, albumin level, alkaline phosphatase level, glucose level, hemoglobin level, hematocrit level, A1C level, etc. In certain examples, the computing system can access multiple blood test results of the prospective client to determine the client's sample information over time and any improvements or detriments. The health lab database 180 can be operated or otherwise utilized by a health care provider of the client 197, such as the client's 197 hospital service (e.g., Blue Cross/Blue Shield, Kaiser Health, etc.), a lab vendor (e.g., Quest, LabCorp, etc.), or a data aggregator (e.g., ExamOne, LexisNexis, Millman, etc.). The client's 197 health care provider typical performs period clinical health tests on the client 197, such as blood work, biopsies, urine tests, stool test, etc., to determine whether certain test levels are abnormal in accordance with medical industry standards.

According to examples provided herein, the health lab data engine 140 can provide stricter standards to identify clients 197 of exceptional health and longevity that deserve lower cost and/or higher quality risk-mitigation product access. These stricter standards can comprise more ideal ranges for each received data set in the clinical health lab data of the client 197. The health lab data engine 140 can execute the classifier model 142 to classify each aspect of the client's 197 health lab data within a range specific to that aspect. Thus, the classifier model 142 can classify the client's 197 creatinine level in accordance with the ideal range for creatinine levels. For each aspect of the client's 197 health lab data, the classifier model 142 can classify the aspect to determine whether the aspect is within the ideal range. Based on the classifications of the client's 197 health lab data by the classifier model 142, the health lab data engine 140 can determine whether the client 197 satisfies each criterion for each aspect of the health lab data. In one example, the output of the health lab data engine 140 can comprise a health or mortality risk score. In variations, the health lab data engine 140 can output a mortality table with the client classified in a particular grouping or tier of the mortality table (e.g., a percentile based on age and gender).

In certain variations, the historical data (e.g., prescription, medical or insurance claims, and diagnosis information) and health lab data can be processed in combination by the health tier filter 130 and health lab data engine 140 to make an overall determination of risk mitigation tier, risk score, or underwriting class for the client 197. In some examples, the results may indicate that the client 197 is currently in an incorrect tier or underwriting class compared to when looking at each data element in isolation, and/or should be downgraded from a current tier or underwriting class. In further examples, the results may indicate that the client 197 should be declined from coverage outright.

As described herein, the classifier model 142 can use historical sample data from a control group of past patients, and correlate those data to the historical medical claims and prescription information of the past patients, the classifier model 142 can generate an ideal range or set of ranges for each clinical lab data set (e.g., a healthy cholesterol range, HDL ratio, creatinine level, etc.). Based on the historical data, these ideal ranges can correlate to prospective clients 197 that will qualify for the top tier or underwriting class. As such, the classifier model 142 can determine the ideal ranges for each clinical lab data set as a proprietary and customized set of health data ranges.

In further examples, the health lab data engine 140 can receive activity data from an activity monitoring device (e.g., a wearable computing device, such as a wrist-worn device, or the client's mobile computing device 190) that can indicate information such as the prospective client's 197 heart rate, the number of steps taken throughout the day, calories burned, sleep scores, etc. Such a device may include sensors, such as an IMU, pulse rate detector, etc., that can measure heart rate, steps taken, calories burned, and the like. The health lab data engine 140 can further process the activity data of the prospective client 197 (e.g., for a previous month or multiple previous months) to determine an activity level and/or sleep level of the prospective client 197 as compared to an activity range for humans in general. The classifier model 142 can further classify the prospective client 197 in each aspect of the activity data and/or sleep score data (e.g., 85th percentile in steps taken per day) and incorporate the prospective client's 197 activity and/or sleep levels with the prospective client's 197 clinical lab data set information in determining a health or mortality score for the prospective client 197.

As described herein, the health lab data engine 140 can also factor in a cognitive risk score of the client 197 if the client 197 chooses to take a cognitive assessment test (“CAT”) that comprises filtered questions and assertions (e.g., a multiple-choice quiz) that test various cognitive health metrics of the users, such as spatial reasoning, motor function, word-finding and language processing ability, and time to completion for each question/assertion as well as the overcall CAT. As such, CAT performance of the client 197 can enable the computing system to generally measure the client's 197 cognitive well-being by digitally assessing memory and recall, word finding abilities, reaction time and motor function, and the like. Each of the above factors can be factored in to generate an overall health or risk score of the client 197, establish the client's 197 classification in a mortality table, and/or determine morbidity and/or vehicle accident risk for the client 197.

Based on the health or mortality risk score, a risk mitigation policy generator 150 of the computing system 100 can determine to which underwriting class or tier the client 197 belongs. In some aspects, the risk mitigation policy generator 150 can generate an individualized risk mitigation policy package for the client 197 based on the unique information provided from the client 197, such as the historical data, health lab data, activity data, and the like. Thus, based on the classification of the client 197, the risk mitigation policy generator 150 can generate a service customer interface—via the health service app 196 or website—that provides the risk mitigation package for the client 197. In one example, the interface enables the client 197 to engage with a redemption or purchase interface to redeem or purchase the risk-mitigation package or a discount thereof. In additional aspects, the service customer interface and purchase interface can provide the client 197 with customized information concerning the client's 197 own health lab data results (e.g., percentile results for each aspect of the health lab data of the client 197), activity data (e.g., an activity percentile based on the client's 197 age), and/or prescription and claims history.

It is contemplated that prior technical problems involving lack of cross-access to health lab data, lack of synchronization capability with a prospective client's health service provider accounts, and a lack of combined data accumulation, classification, and processing has restrained the risk-mitigation industry from more granular risk assessment of individuals—making the industry reliant on antiquated general classifications of individuals based on basic information such as age, weight, gender, demographics, etc. With the advent of big data and network technology, the ability to rapidly access, combine, and process multiple data sets through minimal user interaction has resulted in a technical solution to the previous norms in the industry. With the implementation of the concepts described herein, risk-mitigation can become more granular and individualized to provide for a more equitable cost structure and distribution of risk-mitigation funds (e.g., insurance charges), and can have an overall effect of higher efficiency and certainty in the health risk-mitigation industry. Furthermore, the techniques described herein may eliminate costs and certain unnecessary steps that currently need to be performed by the prospective client 197, such as making additional medical appointments for lab work, enabling the prospective client 197 to potentially acquire coverage on-demand.

Client Computing Device

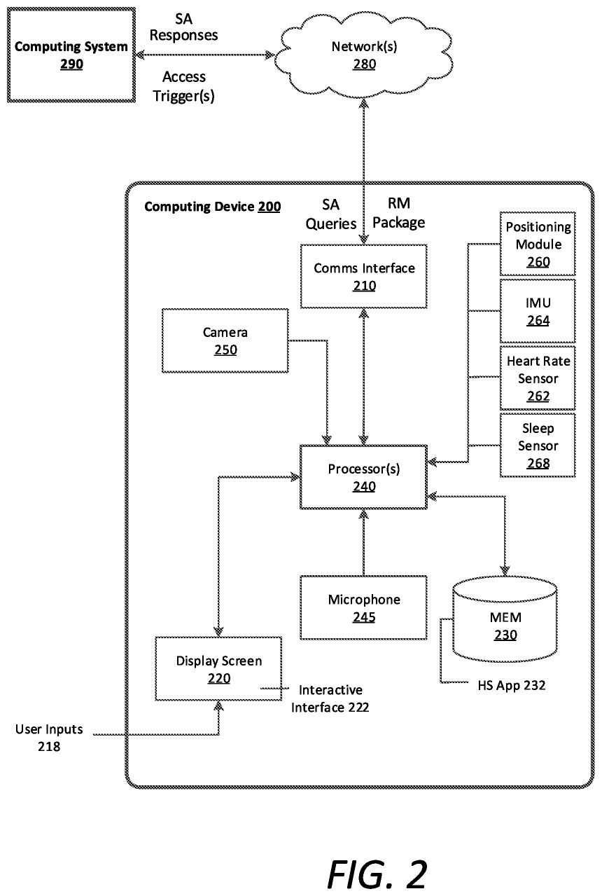

FIG. 2 is a block diagram illustrating an example computing device executing a health service application 232 for communicating with a computing system 290 (e.g., the computing system 100 of FIG. 1), according to examples described herein. In many implementations, the computing device 200 can comprise a mobile computing device, such as a smartphone, tablet computer, laptop computer, personal computer, VR or AR headset device, and the like. In certain examples, the computing device 200 can include telephony features such as a microphone 245, a camera 250, and a communication interface 210 to communicate with the computing system 290 using any number of wireless communication protocols. The computing device 200 can further include a positioning module 260 (e.g., GPS) and an inertial measurement unit 264 that includes one or more accelerometers, gyroscopes, or magnetometers.

In certain aspects, the computing device 200 can store a designated health service application 232 in a local memory 230, and/or the computing device 200 can access the one or more networks 280 via a web browser. In variations, the memory 230 can store additional applications executable by one or more processors 240 of the computing device 200, enabling access and interaction with one or more host servers over the one or more networks 280. In still further examples, the computing device 200 can provide access to the Internet such that a prospective client 197 can access a website of the health and risk mitigation service provided by the computing system 290.

Additionally, the computing device 200 can be operated by a prospective client 197 through execution of the health service application 232. In various examples, the client 197 can select the health service application 232 via a user input 218 on the display screen 220, which can cause the application 232 to be executed by the processor 240. In response, an interactive user interface 222 can be generated on the display screen 220, which can display the various features of the health and risk assessment service provided by the computing system 290.

As provided herein, the application 232 can enable a communication link over one or more networks 280 with the computing system 290, such as the computing system 100 as shown and described with respect to FIG. 1. The processor 240 can generate user interface features 222 using content data received from the computing system 290 over network 280. Furthermore, as discussed herein, the application 232 can enable the computing system 290 to cause the interactive user interface 222 to be displayed on the display screen 220.

In various examples, the computing device 200 may also include activity monitoring systems, such as the IMU 264, heart or pulse rate sensor 262, a sleep quality sensor 268, and the positioning module 260. According to the above examples, the client 197 can interact with the user interface 222 (e.g., via the display screen 220 or through a web interface) to receive the self-assessment queries and provide self-assessment responses to enable the computing system 290 to perform the verification tasks described above. Furthermore, the client 197 can interact with the interface 222 to provide access triggers that enable the computing system 290 to access the historical prescription data, medical claim data, health lab data, and activity data from the activity monitoring devices 260, 262, 264, 268 of the computing device 200. Upon processing the data by the computing system 290, the computing device 200 can receive a risk-mitigation package that comprises content data displayed on the interactive user interface 222

Thus, the interactive user interface 222 can present individualized content specific to the client 197 based on the client's historical information, activity data, and health lab data. When the client 197 qualifies for the top tier risk-mitigation package(s), the interactive interface 222 and provide the information that indicates why the client 197 has qualified (e.g., various percentiles described herein) and a service customer interface to enable the client 197 to view the product details and purchase a selected risk-mitigation package.

Methodology

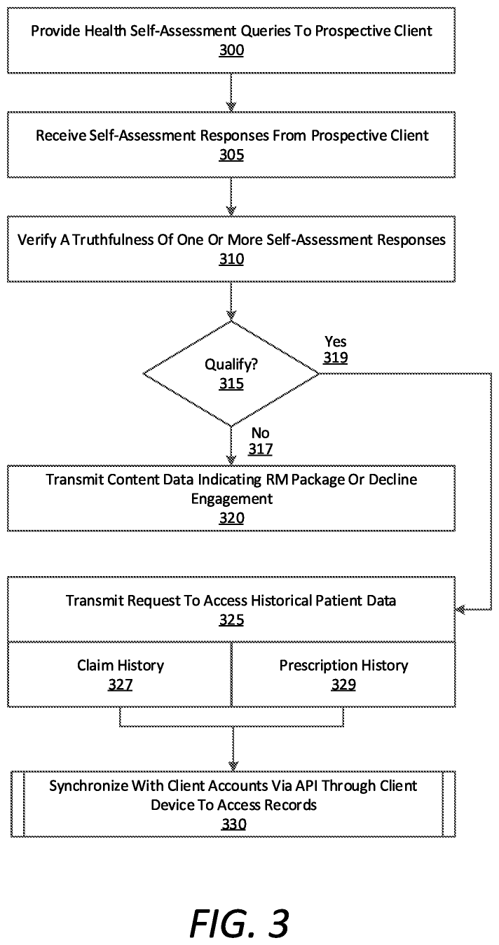

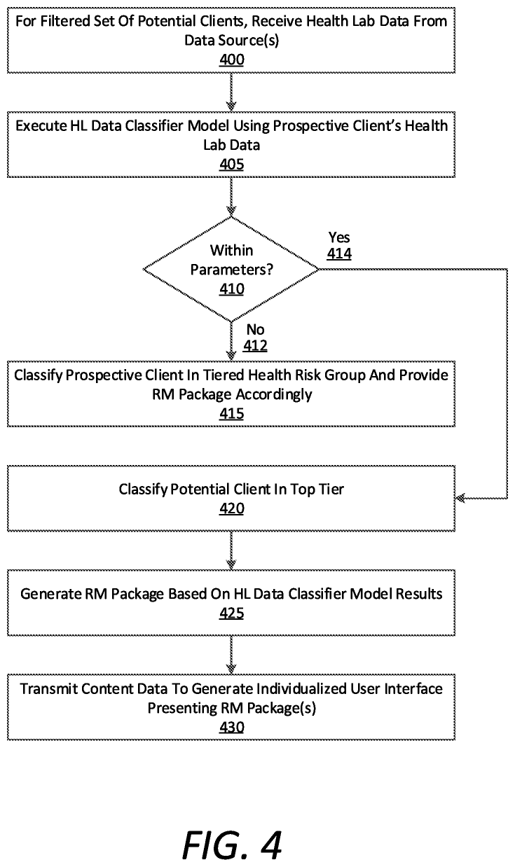

FIGS. 3 and 4 are flow charts describing example methods of verifying self-assessment responses from clients and executing a health data classifier model based on a client filtering process, according to various examples. In the below description of FIGS. 3 and 4, reference may be made to reference characters representing certain features discussed with respect to FIGS. 1 and 2. Furthermore, the below processes described in connection with FIGS. 3 and 4 may be performed by an example computing system 100, 290 as shown and described with respect to FIGS. 1 and 2. Referring to FIG. 3, the computing system 100 can provide a set of self-assessment queries to a prospective client 197 (300), and receive a corresponding set of self-assessment responses from the prospective client 197 (305).

For one or more of the self-assessment responses, the computing system 100 can verify truthfulness of the self-assessment response(s) (e.g., using location data from the client's 197 computing device 190) (310). For example, if the client 197 is asked where the client 197 currently resides, the computing system 100 can utilized the location data as a verification tool for the prospective client's 197 response. The computing system 100 may then determine, based on the self-assessment responses, whether the prospective client 197 qualifies for a next step in the filtering process based on a set of basic criteria (315). As described herein, such criteria may involve any combination of whether the prospective client 197 resides in a nursing home, whether the prospective client 197 is a smoker, a height to weight ratio or BMI of the prospective client 197, an age of the prospective client 197, activity and health information of the prospective client 197, and the like.

If the prospective client 197 does not qualify (317), the computing system 100 can transmit content data indicating a risk-management package (e.g., a life insurance package, a Medicare supplement insurance package, etc.) for which the prospective client 197 does qualify, or decline further engagement (320). However, if the prospective client 197 qualify for a next step based on the initial criteria, the computing system 100 can transmit an access or synchronization request to receive historical patient data of the prospective client 197 (325). As provided herein, the historical data can comprise the prospective client's 197 medical claim history (327) and prescription history (329). In certain examples, the computing system 100 can provide an icon or graphical button that, when selected, provides the necessary access permissions that enables the computing system 100 to synch with one or more health-related accounts of the prospective client 197 (e.g., a health care provider account, a prescription provider account, a health insurance account, mymedicare.gov account, etc.). Upon receiving a trigger indicating the prospective client's 197 selection of the graphical indicator or icon, the computing system 100 can synchronize with the client's health account(s) through the client's computing device 190 to access the records, to determine if the prospective client 197 qualifies for a next tier or underwriting class (330).

If the prospective client 197 qualifies for a next step in the process based on the historical prescription and medical claims data (e.g., based on a second set of criteria comprising total number and/or cost of prescriptions and/or medical claims of the prospective client 197), the computing system 100 can then transmit a request for access to the prospective clients 197 health lab data. Thus, at this stage, the computing system 100 communicates with a filtered set of prospective clients 197 that have been verified at an initial step, and screened based on historical data. All other prospective clients 197 have either been declined or have been provided with lower-tiered risk-mitigation packages. Referring to FIG. 4, the computing system 100 may receive health lab data of the filtered set of clients 197 from one or more health lab databases 180 (400). As described herein, the health lab data can correspond to blood tests, urine samples, body tissue samples, etc., and can provide certain aspect levels of the prospective client 197, such as total cholesterol level, high-density lipoprotein (HDL) level, HDL ratio, triglycerides level, creatinine level, albumin level, alkaline phosphatase level, glucose level, hemoglobin level, hematocrit level, A1C level, etc.

In various examples, the computing system 100 can execute a heath lab data classifier model 142 using the prospective client's 197 health lab data (405). The classifier model 142 can comprise a custom set of ideal ranges or parameters for each of the health aspect levels (e.g., proprietary ranges that are predictive of low morbidity and/or mortality risk), and can execute to determine whether the prospective client's 197 health aspect levels are within these ranges or parameters (410). If the prospective client's 197 health aspect levels are outside the parameters (412), then the computing system 100 classifies the prospective client 197 within the normal or lower tiered health/mortality risk group, and provides a risk-mitigation package accordingly, or declines further engagement (415). However, if the health aspect levels of the prospective client 197 are within the parameters (414), then the computing system 100 can classify the client 197 in the top tier of risk-mitigation (420). In various examples, this means that the prospective client 197—an exceptionally healthy senior citizen—can be provided with access to far lower cost and/or customized risk-mitigation products or packages as opposed to being forces to purchase normal, higher cost packages based on antiquated factors, such as age.

It is contemplated that the historical data (e.g., prescription, medical claims, and/or diagnosis information) and health lab data can be processed in combination or simultaneously by the computing system 100 to make an overall determination of risk mitigation tier or underwriting class for the client 197. In some examples, the results may indicate that the client 197 is currently in an incorrect tier or underwriting class compared to when looking at each data element in isolation, and/or should be downgraded from a current tier or underwriting class. In further examples, the results may indicate that the client 197 should be declined from coverage. Thus, in such examples, the computing system 100 can make a more holistic determination with regard to underwriting class or risk-mitigation tier for the prospective client 197. For example, prescription data for the client 197 may normally result in a decline, but when the client's 197 lab data is taken into account, the computing system 100 may approve the client 197 for a better risk-mitigation class than when compared to only having access to the client's 197 prescription data.

It is further contemplated that additional factors may be utilized by the computing system to determine underwriting class, such as credit data, criminal history, driving records, data corresponding to prior insurance application history (e.g., from Medical Information Bureau data (MIB)), dental records, smoker predictive models, systolic and diastolic blood pressure, and the like. Furthermore, the risk-mitigation tiers or classes may be continuous or discrete. For example, current implementations use a limited number of underwriting classes for customers (e.g., three tiers involving low risk, medium risk, and high risk). According to examples provided herein, the computing system 100 can implement continuous tiering involving far more granular risk-mitigation tiers or underwriting classes (e.g., one hundred or more).

Upon determining a tier or class for the client 197, the computing system 100 may generate one or more risk-mitigation packages for the client 197 based on the health lab data classifier model 142 results (425). In some examples, the risk mitigation package(s) can be individually tailored for the client 197, with customized coverage and pricing based on the specific health aspect levels of the client 197. In variations, the top tiered risk mitigation package(s) can comprise added coverage and/or discounted pricing that is general to all who qualify based on the classifier model 142 results. Upon generating the risk-mitigation package(s), the computing system 100 may then transmit content data to the computing device 190 of the client 197 (e.g., via the executing health service app 196 or over a web browser) that causes the computing device 190 to present an interactive service customer interface 222 that the client 197 can interact with to view and/or purchase the offered risk-mitigation package(s) (430).

Hardware Diagram

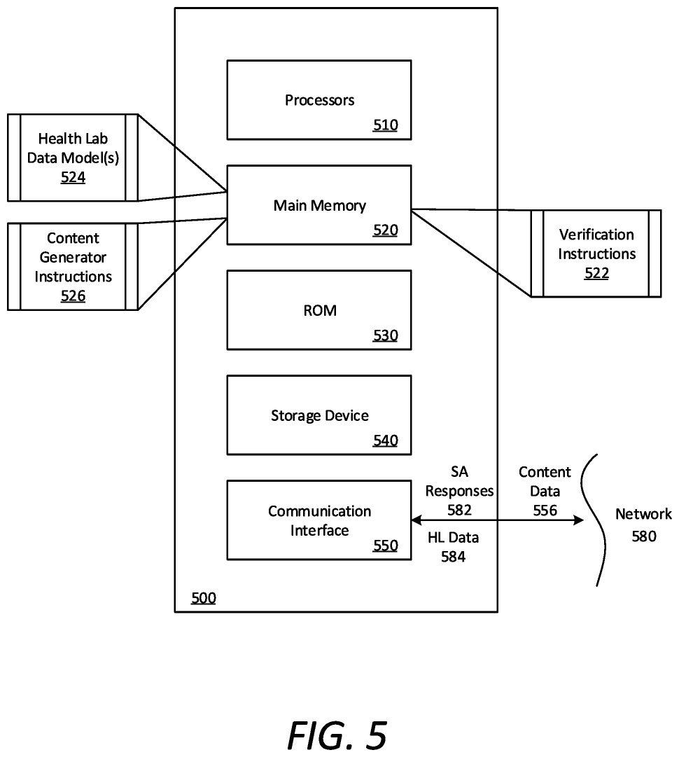

FIG. 5 is a block diagram that illustrates a computer system 500 upon which examples described herein may be implemented. A computer system 500 can be implemented on, for example, a server or combination of servers. For example, the computer system 500 may be implemented as part of a network-based service, such as described in FIGS. 1 through 4. In the context of FIG. 1, the computing system 100 may be implemented using a computer system 500 such as described by FIG. 5. The computer system 100 may also be implemented using a combination of multiple computer systems as described in connection with FIG. 5.

In one implementation, the computer system 500 includes processing resources 510, a main memory 520, a read-only memory (ROM) 530, a storage device 540, and a communication interface 550. The computer system 500 includes at least one processor 510 for processing information stored in the main memory 520, such as provided by a random-access memory (RAM) or other dynamic storage device, for storing information and instructions which are executable by the processor 510. The main memory 520 also may be used for storing temporary variables or other intermediate information during execution of instructions to be executed by the processor 510. The computer system 500 may also include the ROM 530 or other static storage device for storing static information and instructions for the processor 510. A storage device 540, such as a magnetic disk or optical disk, is provided for storing information and instructions.

The communication interface 550 enables the computer system 500 to communicate with one or more networks 580 (e.g., cellular network) through use of the network link (wireless or wired). Using the network link, the computer system 500 can communicate with one or more computing devices, one or more servers, and/or one or more databases. In accordance with examples provided herein, the executable instructions stored in the memory 520 can include verification instructions 522, a health lab data classifier model 524, and content generator instructions 526.

By way of example, the instructions and data stored in the memory 520 can be executed by the processor 510 to implement the functions of an example computing system 100 of FIG. 1. In various examples, the processors 510 can execute the verification instructions 522 to process self-assessment response from a prospective client to verify truthfulness. The processors 510 can further execute the health lab data model 524 to classify the health aspect levels of a prospective client in a proprietary range set to determine whether the prospective client qualifies for a top tier of risk mitigation package(s). In still further examples, the processors 510 can execute the content generator instructions 526 to generate a risk-mitigation package based on the health or mortality risk of the prospective client 197 as determined through execution of the health lab data classifier.

Examples described herein are related to the use of the computer system 500 for implementing the techniques described herein. According to one example, those techniques are performed by the computer system 500 in response to the processor 510 executing one or more sequences of one or more instructions contained in the main memory 520. Such instructions may be read into the main memory 520 from another machine-readable medium, such as the storage device 540. Execution of the sequences of instructions contained in the main memory 520 causes the processor 510 to perform the process steps described herein. In alternative implementations, hard-wired circuitry may be used in place of or in combination with software instructions to implement examples described herein. Thus, the examples described are not limited to any specific combination of hardware circuitry and software.

It is contemplated for examples described herein to extend to individual elements and concepts described herein, independently of other concepts, ideas or systems, as well as for examples to include combinations of elements recited anywhere in this application. Although examples are described in detail herein with reference to the accompanying drawings, it is to be understood that the concepts are not limited to those precise examples. As such, many modifications and variations will be apparent to practitioners skilled in this art. Accordingly, it is intended that the scope of the concepts be defined by the following claims and their equivalents. Furthermore, it is contemplated that a particular feature described either individually or as part of an example can be combined with other individually described features, or parts of other examples, even if the other features and examples make no mentioned of the particular feature. Thus, the absence of describing combinations should not preclude claiming rights to such combinations.

What is claimed is:

1. A computing system implementing a risk-assessment service, comprising:
a network communication interface to communicate, over one or more networks, with computing devices of users of the risk assessment service;
one or more processors; and
a memory resource storing instructions that, when executed by the one or more processors, cause the computing system to:
receive, over the one or more networks, an inquiry from a computing device of a prospective client;
based at least in part on the inquiry, access, over the one or more networks, health lab data of the prospective client from one or more health lab data sources;
upon accessing the health lab data, execute a health lab data classifier to determine whether a set of health aspect levels indicated in the health lab data are within a set of health aspect ranges; and
upon determining that the set of health aspect levels indicated in the health lab data of the prospective client are within the set of health aspect ranges, transmit, over the one or more networks, content data to the computing device of the prospective client, the content data causing the computing device of the prospective client to present an interactive user interface enabling the prospective client to redeem a benefit for at least one risk-mitigation package, the benefit corresponding to a particular tier of the risk-assessment service.

2. The computing system of claim 1, wherein the health aspect levels indicated in the health lab data comprises at least one of a total cholesterol level, a high-density lipoprotein (HDL) level, an HDL ratio, a triglycerides level, a creatinine level, an albumin level, an alkaline phosphatase level, a glucose level, a hemoglobin level, a hematocrit level, ALT level, AST level, total bilirubin, BUN/creatinine ratio, urea nitrogen (BUN) level, albumin/globulin ratio, EGFR level, bilirubin level, vitamin D level, C-reactive protein level, troponin I level, potassium level, hepatitis C condition, urine protein test result levels, cystatin C level, or an A1C level.

3. The computing system of claim 1, wherein the executed instructions further cause the computing system to:
receive, over the one or more networks, location data indicating a current location of the prospective client from the computing device of the prospective client;
in response to receiving the inquiry, transmit, over the one or more networks, a set of self-assessment queries to the computing device of the prospective client; and
receive, over the one or more networks, a corresponding set of self-assessment responses from the computing device of the prospective client.

4. The computing system of claim 3, wherein the executed instructions further cause the computing system to:
execute verification logic on at least one self-assessment query of the corresponding set of self-assessment queries to verify truthfulness of the at least one self-assessment query.

5. The computing system of claim 4, wherein the at least one self-assessment query comprises a home address query to provide a current address of the prospective client, and wherein execution of the verification logic causes the computing system to determine the truthfulness of the home address query by comparing the current location of the prospective client with a home address response provided by the prospective client to the home address query.

6. The computing system of claim 3, wherein the executed instructions further cause the computing system to: based on the corresponding set of self-assessment responses, determine whether the prospective client qualifies for a next step in a risk-assessment process of the risk-assessment service using a set of initial criteria; and
based at least in part on the prospective client qualifying for the next step, generate, on the interactive user interface displayed on the computing device of the prospective client, a graphical feature that, when selected, provides authorization to access to historical health-related data of the prospective client.

7. The computing system of claim 6, wherein the set of initial criteria corresponds to at least one of: whether the prospective client resides in a nursing home, whether the prospective client is a smoker, a height to weight ratio of the prospective client, a BMI of the prospective client, an age of the prospective client, or activity information of the prospective client.

8. The computing system of claim 6, wherein the historical health-related data comprises historical prescription information, historical medical claims information, and historical medical diagnosis information of the prospective client.

9. The computing system of claim 8, wherein the executed instructions further cause the computing system to:
receive, over the one or more networks, trigger data from the computing device of the prospective client, the trigger data indicating that the prospective client has selected the graphical feature and provided authorization to access the historical health-related data; and
in response to receiving the trigger data, transmit, over the one or more networks, an access request to at least one historical health database to receive the historical prescription information, the historical medical claims information, and the historical medical diagnosis information of the prospective client.

10. The computing system of claim 9, wherein the executed instructions further cause the computing system to:
determine whether the historical prescription information, the historical medical claims information, and the historical medical diagnosis information of the prospective client satisfy a second set of criteria;
wherein the executed instructions cause the computing system to transmit the access request for the health lab data of the prospective client in response to determining that the historical prescription information, the historical medical claims information, and the historical medical diagnosis information of the prospective client satisfy the second set of criteria.

11. The computing system of claim 10, wherein the second set of criteria corresponds to a total number or a total cost related to the historical prescription information, the historical medical claims information, and the historical medical diagnosis information of the prospective client.

12. The computing system of claim 1, wherein the executed instructions further cause the computing system to:
receive, over the one or more networks, activity data from an activity monitoring device of the prospective client;
wherein the executed instructions cause the computing system to further determine that the prospective client qualifies for the particular tier of the risk-assessment service based on the activity data.

13. The computing system of claim 12, wherein the activity data indicates at least one of a heart rate, a number of steps taken throughout a day, a pace of exercise, a distance traveled, a number of calories burned, or a sleep score of the prospective client over a period of time or from a single activity.

14. The computing system of claim 12, wherein the activity monitoring device is a wearable device, the wearable device comprising at least one of a wrist-worn device, a chest-worn device, or an embedded monitoring device in exercise equipment.

15. The computing system of claim 12, wherein the activity monitoring device is the computing device of the prospective client.

16. The computing system of claim 1, wherein the executed instructions further cause the computing system to:
query the prospective client for a home address;
receive a home address response from the prospective client; and
compare the home address response against a database comprising locations of nursing homes and assisted living facilities to determine whether the home address matches a nursing home or assisted living facility location.

17. The computing system of claim 16, wherein the executed instructions cause the computing system to execute fuzzy logic to determine whether the home address matches a nursing home or assisted living facility location, the fuzzy logic including a truth range for each location of nursing home and assisted living facility in the database.

18. The computing system of claim 1, wherein the executed instructions further cause the computing system to:
generate, on the interactive user interface, a prompt for the prospective client to log into a health-related account and provide authorization to access historical health-related data of the prospective client;
receive, over the one or more networks, trigger data indicating that the prospective client has provided authorization to access the historical health-related data; and
in response to receiving the trigger data, access the historical health-related data of the prospective client;
wherein the executed instructions cause the computing system to further determine the particular tier of the risk-assessment service for the prospective client based on the historical health-related data.

19. A non-transitory computer readable medium storing instructions that, when executed by one or more processors, cause the one or more processors to:
communicate, over one or more networks, with computing devices of users of the risk assessment service;
receive, over the one or more networks, an inquiry from a computing device of a prospective client;
based at least in part on the inquiry, access, over the one or more networks, health lab data of the prospective client from one or more health lab data sources;
upon accessing the health lab data, execute a health lab data classifier to determine whether a set of health aspect levels indicated in the health lab data are within a set of health aspect ranges; and
upon determining that the set of health aspect levels indicated in the health lab data of the prospective client are within the set of health aspect ranges, transmit, over the one or more networks, content data to the computing device of the prospective client, the content data causing the computing device of the prospective client to present an interactive user interface enabling the prospective client to redeem a benefit for at least one risk-mitigation package, the benefit corresponding to a particular tier of the risk-assessment service.

20. A computer-implemented method of facilitating a risk assessment service, the method being performed by one or more processors and comprising:
communicating, over one or more networks, with computing devices of users of the risk assessment service;
receiving, over the one or more networks, an inquiry from a computing device of a prospective client;
based at least in part on the inquiry, accessing, over the one or more networks, health lab data of the prospective client from one or more health lab data sources;
upon accessing the health lab data, executing a health lab data classifier to determine whether a set of health aspect levels indicated in the health lab data are within a set of health aspect ranges; and
upon determining that the set of health aspect levels indicated in the health lab data of the prospective client are within the set of health aspect ranges, transmitting, over the one or more networks, content data to the computing device of the prospective client, the content data causing the computing device of the prospective client to present an interactive user interface enabling the prospective client to redeem a benefit for at least one risk-mitigation package, the benefit corresponding to a particular tier of the risk-assessment service.
